(19) United States
(12) Patent Application Publication
(10) Pub. No.: US 2017/0081627 A1
(43) Pub. Date: Mar. 23, 2017

(54) PLATELET ACTIVATION DEVICE HAVING MULTI-CHANNEL BLOOD PASSAGE

(71) Applicant: Good morning Bio Co., Ltd., Incheon (KR)

(72) Inventor: Eui Jae HWANG, Incheon (KR)

(73) Assignee: Good morning Bio Co., Ltd., Incheon (KR)

(21) Appl. No.: 15/310,821

(22) PCT Filed: Feb. 11, 2015
(86) PCT No.: PCT/KR2015/001360

§ 371 Date: Nov. 14, 2016

(30) Foreign Application Priority Data
May 22, 2014 (KR) 10-2014-0061362

(51) Int. Cl. C12M 1/42; C12M 1/00; C12M 1/26; C12M 3/06

(52) U.S. Cl. CPC C12M 35/04; C12M 23/16; C12M 29/14; C12M 33/12

(57) ABSTRACT

The present invention relates to a platelet activation device and, more particularly, to a platelet activation device having a multi-channel blood passage, which applies capillary stress to a biological fluid including platelets, thereby activating the platelets included in the blood. The present invention provides an activation structure having multiple microchannels, the structure comprising at least one blood passage, each of which has the same width and comprises multiple bent portions, wherein, while blood successively passes through the multiple bent portions, physical stress is applied to the platelets included in the blood, thereby inducing activation of the platelets, and ⅓ of the amount of blood is distributed to each blood passage, so as to correspond to the number of blood passages, thereby substantially reducing the number of reciprocating movements of blood and shortening the platelet activation time to a large extent.

### TECHNICAL FIELD

The present invention relates to a platelet activation device, and more particularly, to a platelet activation device having a multi-channel blood passage that applies capillary stress to a biological fluid including platelets to activate the platelets contained in the blood.

### BACKGROUND ART

Blood carries out various functions. For example, blood delivers oxygen taken by lungs to tissue cells, and transports carbon dioxide from tissues to the outside of lungs. Blood delivers nutrients absorbed by alimentary canal to organs or tissue cells. Blood transports degradation products of tissues, which are unnecessary for the living body, to a kidney and discharges them to the outside of the body. Blood delivers hormones secreted by endocrine glands to acting organs or tissues, and keeps body temperature constant by equally distributing body heat. In addition, blood destroys or detoxifies invading germs or foreign substances.

Such blood is used as an important index for determining various diseases or health conditions. Platelets abundantly containing growth factors in the blood are used for therapeutic purposes. Blood is composed of red blood cells, white blood cells, and platelets. The platelets mainly exist in plasma. The plasma is divided into platelet rich plasma (PRP) and platelet poor plasma (PPP). The PRP is transplanted into a pain area, in particular, knees, ligaments, muscles, and the like, and stimulates stem cells to help creating cells. As such, the PRP has been used for therapeutic purposes.

It has been known that platelets in the PRP are 2-7 times more than platelets in general blood, and growth factors created and secreted from the platelets promote cell regeneration of a wounded area, thereby helping wound healing and regeneration of cells or tissue. To more improve the effects of the PRP, a method in which a chemical substance such as calcium in Korea and collagen and thrombin in foreign countries or platelet activating protein is added to activate platelets in advance so that the platelets secrete the growth factors, and then, the injection is administered has been used in clinical tests (Lacoste E et al., J Periodntol. 74(10):1498-507(2003)).

However, if calcium is injected into cartilage, it signifies nothing because calcium is neutralized. If calcium is injected into subcutaneous fat, side effects such as blush, rash, and pain induction occur. Also, since thrombin and collagen directly act to coagulate blood, if thrombin and collagen are injected into patients without being completely separated, thrombin and collagen may act as potential risk factors. Thus, there is a need to improve the method of adding these substances. A report in which physical stress is applied to active platelets, instead of adding a chemical activation factor, is disclosed in “T W Chow et al. Blood, 80:113-120 (1992)”. Also, Korean Patent Application Publication No. 10-2012-0129779 (published on Nov. 28, 2012) has been proposed based on the report.

Korean Patent Application Publication No. 10-2012-0129779 discloses a platelet activation device including: a passage which has an inner diameter of 2 mm or less and through which capillary stress is applied to a biological fluid containing platelets; an accommodation part for the passage; and at least one entrance which is formed in both distal ends of the accommodation part and through which the biological fluid is injected or discharged. The platelet activation device obtains activated platelets by repeatedly passing through the capillary passage having a relatively short length.

However, in Korean Patent Application Publication No. 10-2012-0129779, the passage has a structure in which a narrow region and a wide region are alternately formed, and the platelets are activated therethrough. In such a structure, since a plurality of patterns have to be alternately formed in the fine passage, it may be difficult to manufacture the passage. Also, since the passage is provided as a single channel, the number of reciprocating movements of blood inevitably increases according to an amount of blood so as to achieve the satisfactory activation of the platelets. Consequently, it takes a long time to activate the platelets.

### CITATION LIST

### Patent Literature

(Patent Document 1) KR 10-2012-0129779 A (Nov. 28, 2012)

### SUMMARY OF INVENTION

### Technical Problem

Therefore, the present invention has been made in an effort to solve the problems of the related art, and an object of the present invention is to provide a platelet activation device having a multi-channel blood passage, which has a simple structure as compared to the related art, whereby the platelet activation device is easy to manufacture and is capable of reducing the platelet activation time.

### Solution to Problem

According to an aspect for achieving the above object, a platelet activation device having a multi-channel blood passage includes: a main body including one side having an injection hole through which blood is injected, the other side having a discharge hole through which the blood is discharged, and a coupling groove disposed between the one side and the other side; and an activation structure inserted into the coupling groove of the main body and airtightly fused and coupled to the coupling groove, the activation structure having a bottom surface on which multiple microchannels are disposed in parallel with the same width, so that the blood injected through the injection hole passes through the multiple microchannels and are discharged through the discharge hole 12-3a, wherein the multiple microchannels may include at least one blood passage having the same width, and the blood passage includes a plurality of bent portions.

Preferably, each of the plurality of bent portions may have an angle of 110° to 135°.

Preferably, the blood passage may have a structure in which one side and the other side thereof are formed on the same line and four bent portions are formed in a central portion thereof.

Preferably, the platelet activation device blood may further include an upper cover covering an upper portion of the activation structure and a lower cover coupled to the upper cover to cover a lower portion of the main body, in a state in which the activation structure is airtightly fused and coupled to the coupling groove of the main body.

### Advantageous Effects of Invention

As described above, the present invention provides the activation structure having the multiple microchannels, which include three blood passages having the same width, wherein each of the blood passages has four bent portions each having an angle of 110° to 135° in an extending direction thereof. Accordingly, while the blood successively passes through the four bent portions, the physical stress is applied to the platelets contained in the blood to induce the activation of the platelets, and ⅓ of the amount of blood is distributed to each of the blood passages, so as to correspond to the number of blood passages, thereby substantially reducing the number of reciprocating movements of blood and shortening the platelet activation time to a large extent.

### BRIEF DESCRIPTION OF DRAWINGS

FIG. 1 is an assembled perspective view of a platelet activation device having a multi-channel blood passage according to an embodiment of the present invention.

FIG. 2 is an exploded perspective view of the platelet activation device of FIG. 1.

FIG. 3 is a plan view of the platelet activation device of FIG. 1.

FIG. 4 is cross-sectional views respectively taken along lines A′-A″ and B′-B″ of FIG. 3.

FIG. 5 is a bottom view of an activation structure according to the present invention.

FIG. 6 is a view of a main body according to the present invention.

FIG. 7 is a view of the activation structure according to the present invention.

FIG. 8 is a view of an upper cover and a lower cover according to the present invention.

FIG. 9 is a view illustrating a state in which the upper cover and the lower cover of FIG. 8 are assembled with each other.

### DESCRIPTION OF REFERENCE NUMERALS

10: platelet activation device
11: activation structure
12: main body
12-1: first coupling part
12-2: second coupling part
12-3: third coupling part
13: upper cover
14: lower cover

### DESCRIPTION OF EMBODIMENTS

Advantages and features of the present invention and implementation methods thereof will be clarified through the following embodiments described with reference to the accompanying drawings. The present invention may, however, be embodied in different forms and should not be construed as limited to the embodiments set forth herein.

In this specification, these embodiments are provided so that this disclosure will be thorough and complete, and will fully convey the scope of the present invention to those skilled in the art. Also, the present invention is only defined by the scope of claims. Accordingly, in some embodiments, well-known components, well-known device operations, and well-known techniques will not be described in detail to avoid ambiguous interpretation of the present invention.

Throughout this disclosure, like reference numerals are used to refer to like elements. Also, in the following description, the technical terms are used (mentioned) only for explaining specific embodiments and are not intended to limit the present invention. In this specification, the terms of a singular form may include plural forms, unless stated referred to the contrary. As used herein, the singular forms “a,” “an” and “the” are intended to include the plural forms as well, unless the context clearly indicates otherwise.

It will also be understood that the terms “comprises”, “includes”, and “has”, when used herein, specify the presence of stated elements or operations, but do not preclude the presence or addition of other elements or operations, unless otherwise defined.

Unless otherwise defined, all terms including technical and scientific terms used herein have the same meaning as commonly understood by those skilled in the art to which the present invention pertains. It will be further understood that terms, such as those defined in commonly used dictionaries, should be interpreted will not be interpreted in an idealized or overly formal sense unless expressly so defined herein.

Hereinafter, embodiments of the present invention will be described in detail with reference to the accompanying drawings.

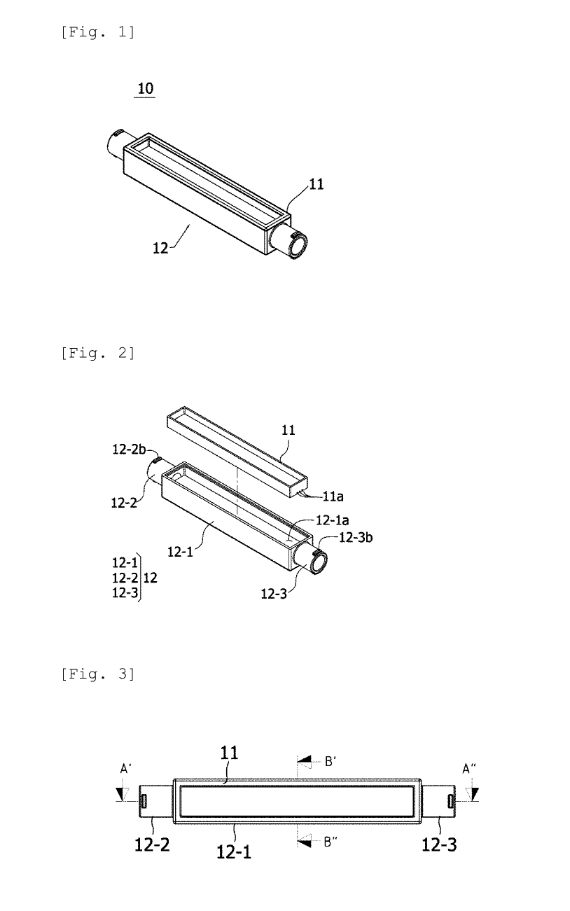

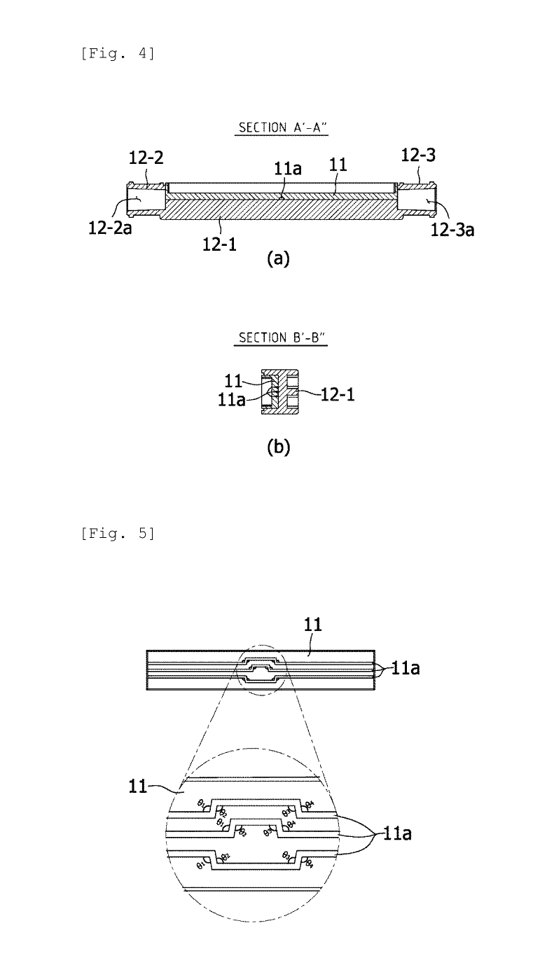

FIG. 1 is an assembled perspective view for explaining a platelet activation device having a multi-channel blood passage according to an embodiment of the present invention, FIG. 2 is an exploded perspective view of the platelet activation device of FIG. 1, FIG. 3 is a plan view of the platelet activation device of FIG. 1, and FIG. 4 is cross-sectional views respectively taken along lines A′-A″ and B′-B″ of FIG. 3.

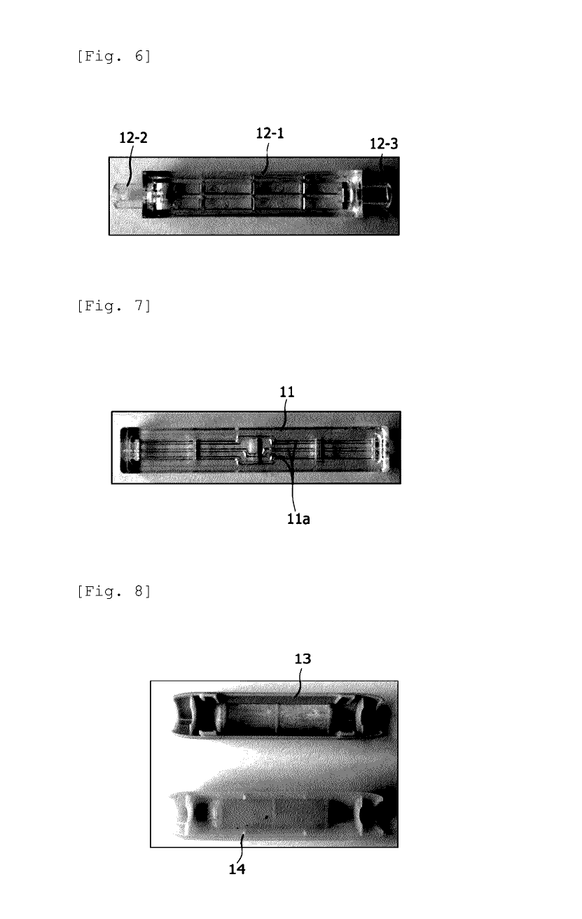

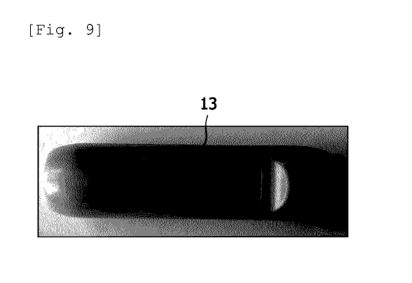

Referring to FIGS. 1 to 4, a platelet activation device 10 having a multi-channel blood passage according to an embodiment of the present invention includes an activation structure 11 and a main body 12. Also, as illustrated in FIGS. 8 and 9, the platelet activation device 10 may further include an upper cover 13 and a lower cover 14.

As illustrated in FIG. 2, the main body 12 includes a first coupling part 12-1 having a coupling groove 12-1a into which the activation structure 11 is inserted and then airtightly fused through a high frequency welding machine, and second and third coupling parts 12-2 and 12-3 which protrude from both sides of the first coupling part 12-1 and are coupled to a blood injector, e.g., a syringe (not shown).

As illustrated in FIG. 2, the first coupling part 12-1 has a rectangular box structure with an upper portion opened. Also, a bottom portion of the coupling groove 12-1a, to which the activation structure 11 is inserted and coupled in a press-fit manner, has a flat surface so as to provide blood passages, through which the blood passes, together with multiple microchannels 11a formed in a bottom surface of the activation structure 11.

As illustrated in FIG. 4, the second and third coupling parts 12-2 and 12-3 have coupling protrusions 12-2b and 12-3b on longitudinal ends so as to be coupled to the blood injector, respectively. An injection hole 12-2a and a discharge hole 12-3a, which communicate with the multiple microchannels 11a, are provided in the second and third coupling parts 12-2 and 12-3, respectively.

FIG. 5 is a bottom view for explaining the activation structure according to the present invention.

Referring to FIGS. 2 and 5, the activation structure 11 may be made of a transparent synthetic resin material so as to recognize a blood flow. The activation structure 11 is inserted into the coupling groove 12-1a of the main body 12 and then coupled to the coupling groove 12-1a in a state of being airtightly fused through the high frequency welding machine, and the multiple microchannels 11a are disposed in parallel with the same width on the bottom surface of the activation structure 11, so that the blood injected through the injection hole 12-2a passes through the multiple microchannels 11a and are discharged through the discharge hole 12-3a.

As illustrated in FIG. 5, the multiple microchannels 11a include three blood passages, and each of the three blood passages includes four bent portions in an extending direction thereof. In this regard, it is preferable that the four bent portions respectively have angles θ1 to θ4 of 110° to 135°.

Also, as illustrated in FIGS. 5 and 7, each of the three blood passages constituting the multiple microchannels 11a has one side and the other side formed on the same line. Here, each blood passage may have a structure in which four bent portions are formed at a central portion thereof or a stair-shaped structure that is gradually stepped upward from one side to the other side thereof. As illustrated in FIGS. 5 and 7, when an injection molding process is performed, one side and the other side may be formed on the same line in terms of a manufacturing process or a spatial margin. Here, it is preferable that the each blood passage has a structure in which the four bent portions are formed at the central portion.

According to the present invention, the multiple microchannels 11a having the three blood channels are provided on the bottom surface of the activation structure 11. Each of the three blood passages includes four bent portions in an extending direction thereof. The four bent portions have angles θ1 to θ4 of 110° to 135° to minimize a pressure applied when the blood is reciprocated so as to apply capillary stress, and ⅓ of the amount of blood is distributed to each of the blood passages, so as to correspond to the number of blood passages and thus apply the stress, thereby implementing the activation for a short time even when the amount of blood is large.

TABLE 1

| | Blood passage | | |
|---|---|---|---|
| | structure | | |
| | | Uneven structure | |
| | | (single channel | |
| | Linear type | disclosed in KR | |
| | (single channel | 10-2012-0129779 A | |
| | having the same | (Nov. 28, | Present |
| | width) | 2012) | invention |
| Number of | 20 times | 15 times | 5-7 times |
| reciprocating | | | |
| movements of | | | |
| blood | | | |

Table 1 above shows the number of reciprocating movements of blood when an activation device having different blood passages was used to activate 5 cc of blood until having the same activation property. In the linear type single channel structure having the same width, the number of reciprocating movements of blood was about 20 times. In the single channel structure disclosed in KR Patent Publication No. 10-2012-0129779 as the related art, the number of reciprocating movements of blood was about 15 times. However, in the multiple microchannel structure having the three blood passages on the bottom surface thereof according to the present invention, the number of reciprocating movements of blood was about 5-7 times. It can be seen from the above result that the number of reciprocating movements of blood is significantly reduced.

In conclusion, the multiple microchannels 11a according to the present invention may include the three blood passages having the same width, and each of the blood passages may have the four bent portions in the extending direction thereof. Also, each of the bent portions may be formed at an angle of 110° to 135°, and thus, the physical stress may be applied to the platelets contained in the blood while the blood passes through the four bent portions to thereby induce the activation of the platelets. In addition, ⅓ of the amount of blood may be distributed to each of the blood passages, so as to correspond to the number of blood passages, thereby substantially reducing the number of reciprocating movements of blood and shortening the platelet activation time to a large extent.

FIG. 6 is a view of the main body according to the present invention, FIG. 7 is a view of the activation structure according to the present invention, FIG. 8 is a view of the upper cover and the lower cover according to the present invention, and FIG. 9 is a view illustrating a state in which the upper cover and the lower cover of FIG. 8 are coupled to each other.

As illustrated in FIGS. 6 and 7, it can be confirmed that the multiple microchannels 11a having the three blood passages are formed on the bottom surface of the activation structure 11 according to the present invention.

As illustrated in FIGS. 8 and 9, the platelet activation device 10 according to the present invention may further include the upper cover 13 and the lower cover 14 so as to protect the activation structure 11 and the main body 12.

The upper cover 13 and the lower cover 14 are made of a synthetic resin material and detachably coupled to each other. The upper cover 13 covers and protects the upper portion of the activation structure 11 in a state in which the activation structure 11 is airtightly fused and coupled to the coupling groove 12-1a of the main body 12, and the lower cover 14 is detachably coupled to the upper cover 13 to cover and protect the lower portion of the main body 12.

As described above, the technical idea of the present invention has been specifically described with reference to the preferred embodiments, but it should be noted that the foregoing embodiments are provided only for illustration while not limiting the present invention. Also, it can be understood by those skilled in the art that various embodiments can be made through combinations of the embodiments of the present invention.

1. A platelet activation device having a multi-channel blood passage, the platelet activation device comprising:
a main body comprising one side having an injection hole through which blood is injected, the other side having a discharge hole through which the blood is discharged, and a coupling groove disposed between the one side and the other side; and
an activation structure inserted into the coupling groove of the main body and airtightly fused and coupled to the coupling groove, the activation structure having a bottom surface on which multiple microchannels are disposed in parallel with the same width, so that the blood injected through the injection hole passes through the multiple microchannels and are discharged through the discharge hole 12-3a,
wherein the multiple microchannels comprise at least one blood passage having the same width, and the blood passage comprises a plurality of bent portions.

2. The platelet activation device blood of claim 1, wherein each of the plurality of bent portions has an angle of 110° to 135°.

3. The platelet activation device blood of claim 1, wherein the blood passage has a structure in which one side and the other side thereof are formed on the same line and four bent portions are formed in a central portion thereof.

4. The platelet activation device blood of claim 1, further comprising an upper cover covering an upper portion of the activation structure and a lower cover coupled to the upper cover to cover a lower portion of the main body, in a state in which the activation structure is airtightly fused and coupled to the coupling groove of the main body.
